(19) United States
(12) Patent Application Publication
(10) Pub. No.: US 2021/0015620 A1
(43) Pub. Date: Jan. 21, 2021

(54) DIGITAL BONE RECONSTRUCTION METHOD

(71) Applicant: SYNTHES GmbH, Oberdof (CH)

(72) Inventors: Steffan DANIEL, Zuchwil (CH); Rhett A. RAPIER, Trimbach (CH); Mario GOETZ, Philippsburg-Rehinsheim (DE); Scott LARSEN, West Chester, PA (US); Horst STEIGNER, Landau/Pfaiz (DE); Alexander WURL, Berlin (DE)

(21) Appl. No.: 16/511,738

(22) Filed: Jul. 15, 2019

(51) Int. Cl. A61F 2/30; A61F 2/28; G06F 3/0481; G06F 3/0484; G06T 17/20; G06T 19/20; B33Y 50/02; B33Y 80/00; B29C 64/393

(52) U.S. Cl. CPC A61F 2/30942; B29L 2031/7532; G06F 3/04815; G06F 3/04845; G06T 17/20; G06T 19/20; B33Y 50/02; B33Y 80/00; B29C 64/393; A61F 2002/2825; A61F 2002/2853; A61F 2002/2892; A61F 2002/30945; A61F 2002/30952; A61F 2002/30985; G06T 2200/24; G06T 2219/2021; G06T 2210/41; A61F 2/28

(57) ABSTRACT

A digital bone reconstruction method that involves receiving medical image data of a bone; displaying on a user interface the bone image; automatically generating, using a processor, a first virtual 3D surface contour of a reconstructed image of the bone having a first geometry and including a plurality of editable control regions; and adjusting at least one of the editable control regions on the first virtual 3D surface contour based on user input to produce a second virtual 3D surface contour of the reconstructed image of the bone having a second geometry.

### TECHNICAL FIELD

This disclosure relates generally to digital bone reconstruction methods. More particularly, this disclosure relates generally to digital bone reconstruction methods for bones with incomplete and/or abnormal anatomy.

### BACKGROUND OF THE INVENTION

Digital bone reconstruction can currently be achieved using a customized design software system. One goal of these software systems is to approximate missing bone geometry, so that an appropriately shaped implant can be created. Principle Component Models (PCMs) and Gaussian Process Models (GPMs) are both common mathematical models used in surface shape modeling. However, such models are not reliable when the bones being analyzed have incomplete or misaligned anatomies. These conditions present error sources for both models. Customized design software systems that overcomes these error sources are needed to optimize treatment for these conditions.

### SUMMARY OF EXEMPLARY EMBODIMENTS

The foregoing advantages of the invention are illustrative of those that can be achieved by the various exemplary embodiments and are not intended to be exhaustive or limiting of the possible advantages that can be realized. Thus, these and other objects and advantages of the various exemplary embodiments will be apparent from the description herein or can be learned from practicing the various exemplary embodiments, both as embodied herein or as modified in view of any variation that may be apparent to those skilled in the art. Accordingly, the present invention resides in the novel methods, arrangements, combinations, and improvements herein shown and described in various exemplary embodiments.

In light of the present need for a customized design software for reconstruction of incomplete and/or abnormal bone anatomies, a brief summary of various exemplary embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

Various embodiments disclosed herein relate to a digital bone reconstruction method including receiving medical image data of a bone; displaying on a user interface the image of the bone, automatically generating, using a processor, a first virtual 3D surface contour of a reconstructed image of the bone having a first geometry and including a plurality of editable control regions, and adjusting at least one of the editable control regions based on user input to produce a second virtual 3D surface contour of the reconstructed image of the bone having a second geometry.

Various embodiments disclosed herein relate to a system for digitally reconstructing a bone including a user interface configured to receive and display medical image data of a bone from an image capture device; and a processor coupled to the user interface, wherein the processor is configured to automatically generate a first virtual 3D surface contour having a first geometry of a reconstructed image of the bone and including a plurality of editable control regions, and adjust the position of at least one of the editable control regions based on user defined input to produce a second virtual 3D surface contour of the reconstructed image of the bone having a second geometry.

In various embodiments, the image of the bone comprises a missing bone portion, a misaligned bone portion, a resected bone portion or combinations thereof.

In various embodiments, the editable control regions include a plurality of splines on the first virtual 3D surface contour of the reconstructed image of the bone. In various embodiments, the splines include a plurality of manipulation handles. In various embodiments, the spacing of the plurality of manipulation handles is dependent upon the radius of curvature of the first virtual 3D surface contour. The manipulation handles may further be linked axially along the length of the first virtual 3D surface contour.

In various embodiments, the step of adjusting at least one of the editable control regions includes dragging the at least one editable control region to an edge of an unreconstructed portion of the bone image.

In various embodiments, the method further involves printing the second virtual 3D surface contour having a second geometry of the reconstructed image of the bone, using a 3D printing device, to produce an implant and administering the implant to a patient.

Various embodiments further relate to a digital bone reconstruction method including receiving medical image data of a bone comprising a missing bone portion; displaying on a user interface a bone image comprising the missing bone portion, automatically generating, using a processor, a first virtual 3D mesh structure contoured to a geometry of the missing bone portion including a plurality of editable control regions, and adjusting at least one of the editable control regions on the first virtual 3D mesh structure based on user input to produce a second virtual 3D mesh structure having a second geometry of the missing bone portion.

In various embodiments, the first and second 3D virtual mesh structures have a circular cross-section and includes outer, inner and interstitial mesh portions.

In various embodiments, the first and second virtual 3D mesh structures further include a plurality of fixation tabs that may be positioned at a proximal or distal end of the first and second virtual 3D mesh structures.

### BRIEF DESCRIPTION OF THE DRAWINGS

In order to better understand various exemplary embodiments, reference is made to the accompanying drawings, wherein:

FIG. 1 is an overview of a conventional process for digital reconstruction of a missing bone anatomy or bone defect;

FIG. 2A illustrates an embodiment of a diagnostic image of a bone having splines with handles;

FIG. 2B illustrates an embodiment of a diagnostic image of a bone showing a vertex calculation for the splines with handles;

FIGS. 3A-3C illustrate manual editing of the diagnostic image of the re-approximated bone using the splines with handles;

FIG. 4 is a flow diagram describing the steps of the digital bone reconstruction method;

FIG. 5 illustrates an embodiment of a mesh structure having an outer mesh portion, an interstitial mesh portion and an inner mesh portion;

FIG. 6 illustrates an embodiment of an inner mesh portion positioned between two bone portions;

FIGS. 7A and 7B illustrate fixation tabs positioned at a distal end of a mesh structure.

### DETAILED DESCRIPTION OF THE INVENTION

Embodiments described herein disclose a digital bone reconstruction method for bones with incomplete and/or abnormal anatomy. Various embodiments described herein disclose a software system to approximate a missing, misaligned, or resected bone geometry so that an appropriately shaped implant may be created. Various embodiments described herein allow for the production of personalized implants for areas of missing, misaligned, or resected bone in various areas of the body. Various embodiments described herein further, more specifically, allow for production of personalized implants for areas of missing, misaligned or resected bone, for example, in the humerus, femur and tibia.

The Principle Component Model (PCM) and Gaussian Process Model (GPM) are both mathematical models used in surface shape modeling. In various embodiments, the digital bone reconstruction method disclosed herein includes utilizing a surface shape modeling system to digitally reconstruct a bone having an incomplete and/or abnormal anatomy. In various embodiments, an incomplete bone anatomy may include a bone having a missing and/or misaligned bone portion. In various embodiments, an abnormal bone anatomy may include a bone having a benign or malignant lesion.

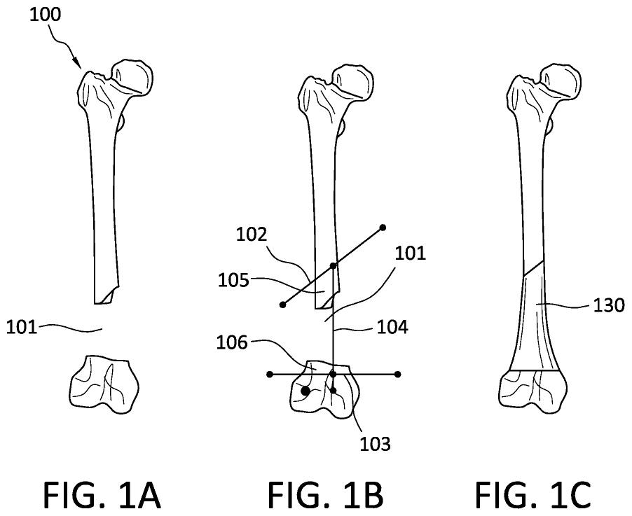

In various embodiments, the digital bone reconstruction method disclosed herein includes utilizing a GPM to digitally reconstruct a bone having incomplete, misaligned, or resected anatomy using customized parameter sets, as shown in FIG. 1A-C, that magnify the mathematical influence of the boney geometry in a region of defect in the GPM.

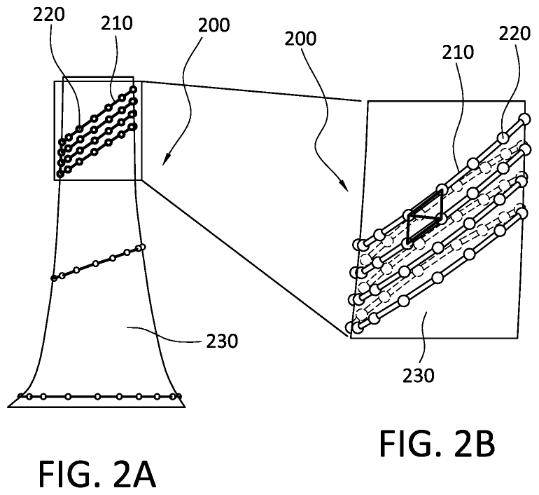

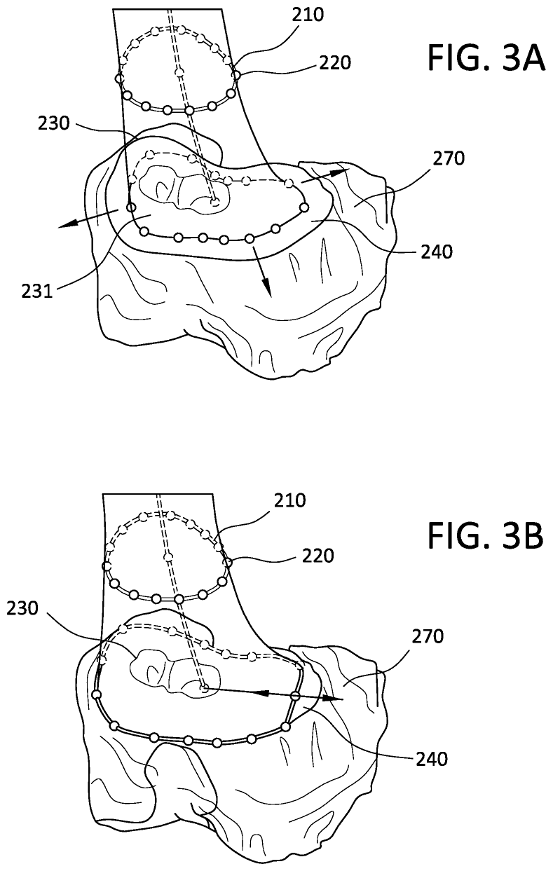

In various embodiments, the method, as shown in FIGS. 2A and 2B, includes further mapping the GPM output using a circumferential spline algorithm that creates splines 210 with virtual handles 220 that allow for direct editing and manipulation of regions of the model. In various embodiments, the method includes a process wherein a first order approximation of the missing, misaligned, or resected bone anatomy is provided by the GPM that, in turn, is allowed to be manipulated by a user to provide a more accurately approximating final geometry, as shown in FIGS. 2-3. FIGS. 2A and 2B illustrate equally spaced splines 210 positioned on the surface of a contour 230. In various embodiments, the contour 230 may represent an approximation of the volumetric bone shape of a missing, misaligned, or resected bone anatomy. In various embodiments, the splines 210 may be spaced on the contour 230 at any distance desired by the user based on clinical need. In various embodiments, additional splines 210 and handles 220 may be added to the surface of the contour 230 at the discretion of the user.

As shown in FIGS. 2A and 2B, the splines 210 follow a central model axis and the ends of the GPM output geometry. Manipulation handles 220 may be positioned at equal distances along each spline 210. In other embodiments, the handles 220 may be spaced at any distance desired by the user based on clinical need. In yet another embodiment, the handles may be spaced based upon the curvature of the spline, i.e., when the radius of curvature is smaller, the handles are more closely spaced, and where the radius of curvature is larger, the handles are less closely spaced. In various embodiments, additional handles 220 may be added to the splines 210 at the discretion of the user. In various embodiments, the splines 210 and handles 220 may be connected to the vertices present in the GPM output model, as shown in FIGS. 2A and 2B. The handles 220 may additionally be linked axially along the length of the model to provide a smooth transition between manipulation points, also as shown in FIGS. 2A and 2B.

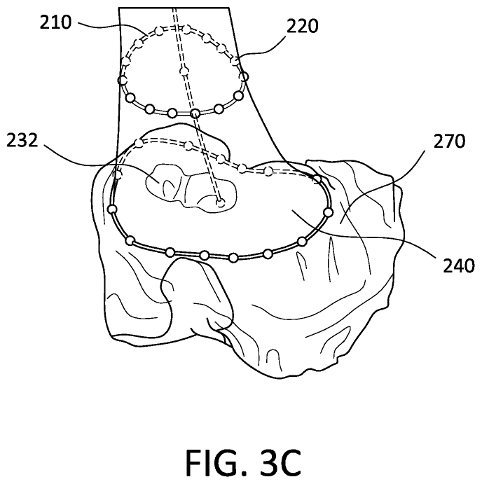

As shown in FIG. 4, in various embodiments, a diagnostic bone image 100 of a bone 101 is generated using an image capture device in a first step 410. Suitable image capture devices may include an X-Ray, MRI, CT device or the like. A processor is then configured to collect user input parameters characterizing an incomplete and/or abnormal bone anatomy in a second step 420. In various embodiments, user input may include an indication of a beginning and end of a missing and/or misaligned bone portion to be filled, as shown in more detail in FIG. 1B. As shown in FIG. 1B, users may input a first horizontal line 102 on a beginning portion 105 of the missing and/or misaligned bone portion and a second horizontal line 103 on an end portion 106 of the missing and/or misaligned bone portion. In various embodiments, users may further input a vertical line 104 connecting the first horizontal line 102 and the second horizontal line 103 that characterizes the size of the missing and/or misaligned bone portion in a longitudinal direction. In some embodiments, the user may virtually cut the bone along the first horizontal line 102 and second horizontal line 103 to produce a smoother surface of each end of the missing bone portion 101. In various embodiments, the processor is then configured to generate a first virtual 3D surface contour 230 of the missing bone portion in a third step 430 and further configured to allow for user manipulation of the first virtual 3D surface contour 230 to desired dimensions and specifications in a fourth step 440 to produce a second virtual 3D surface contour 232. In various embodiments, the method further includes, in a fifth step 450, printing the resulting second virtual 3D surface contour 232 to produce an implant that may be administered to a patient. Suitable implants that may be administered to a patient include bone grafts, bone graft cages, spacers, mesh structures and other implants known to those of skill in the art to treat an incomplete and/or abnormal bone anatomy.

In other embodiments, user input may include an indication of an abnormal bone anatomy, such as a benign or malignant lesion. In such embodiments, the processor may then be configured to automatically resect the bone according to a user-defined resection plan to generate an incomplete bone anatomy. The processor may then be configured to generate a first virtual 3D surface contour of the incomplete bone anatomy and further configured to allow for user manipulation of the first virtual 3D surface contour to desired dimensions and specifications to produce a second virtual 3D surface contour of the incomplete bone anatomy.

In various embodiments, the processor is a hardware device for executing software, particularly that which is stored in memory. The processor may be any custom made or commercially available processor, a central processing unit (CPU), an auxiliary processor among several processors associated with a computer, a semiconductor-based microprocessor (in the form of a microchip or chip set), a macroprocessor, or generally any device for executing software instructions.

In various embodiments, for example, as shown in FIGS. 2A and 2B, the processor may be configured to generate a contour 230 that includes splines 210 and handles 220 on its surface, wherein the splines 210 and handles 220 may allow a user to manipulate the dimensions of contour 230 to a more accurate geometry of a missing and/or misaligned bone portion. In various embodiments, the processor may further be configured to allow the user to zoom into various locations of the contour 230 to further optimize the fit of the contour 230 to the missing and/or misaligned bone portion, as shown in FIGS. 3A-3C.

FIGS. 3A-3C further illustrate an embodiment of a method of manipulation of a distal end 231 of the first virtual 3D surface contour 230 by the user that includes dragging the splines 210 and handles 220 in an outward direction in order to align the edges of the distal end 231 of the first virtual 3D surface contour 230 with the desired edges of the distal bone portion 270 to form a second virtual 3D surface contour 232 completely aligned to a bone face 240 of the distal bone portion 270. In various embodiments, the handles 220 may be configured to be dragged together along with other adjacent handles until all edges of the distal end 231 of the first contour 230 are aligned with the desired edges of the bone face 240 of the distal bone portion 270.

Various modes of selecting and moving the handles may be used. For example, the set of handles 220 that surround the outer surface of the bone may be selected to be moved all at once. In such a case, moving one handle in or out along a radial direction will cause all of the handles to move in the same way. In this case, the external shape of the set of points may be maintained but scaled to a desired size all at once. In another mode, a set of points may be selected and moved as a group. In yet another mode, the number of adjacent handles that may be moved along with a specific handle may be selected and such movement may be such that a smooth transition is made along the handles based upon the movement of the one handle. Other modes allowing for modification of multiple handles at once may also be used.

In FIGS. 3A and 3B, there is an upper set of handles 220 and a lower set of handles 240. After the lower set of handles 240 are moved so that they follow the desired contour of the bone, the upper set of handles 220 may then be adjusted manually by the user to provide sufficient structure for the replacement bone. Also, such adjustment may be done automatically and then further adjusted manually as needed.

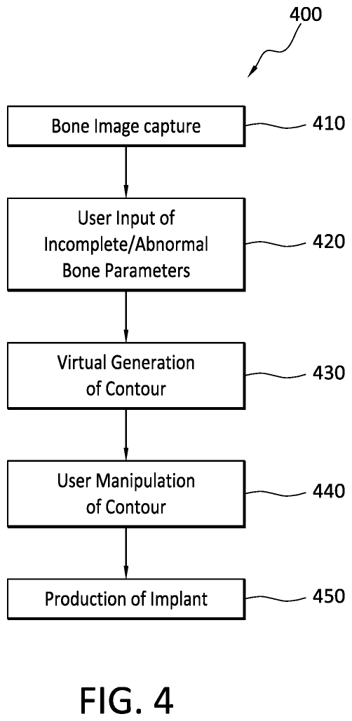

FIG. 5 illustrates how an embodiment of a contour 230 is implemented in the form of a mesh structure 530. In various embodiments, the mesh structure 530 is generated using the model described herein, and may be configured to include a consistent mesh window size within specific regions of the implant structure. In various embodiments, the mesh structure 530 utilizes three separate mesh portions to map surfaces and volumes separately, wherein the three separate mesh portions interface in a repeatable and controlled manner. As shown in FIG. 5, the three separate mesh portions include an outer mesh 540, an inner mesh 550, and an interstitial mesh 560.

In various embodiments, the outer mesh 540 is configured to be contoured to a digitally reconstructed bone surface. The outer mesh 540 may include circumferential struts 541 that may be used to trace an outer cross-sectional shape of the bone geometry. The circumferential struts 541 may be equally spaced, using a static spacing parameter, along the axis of the bone, resulting in a series of rings along the length of the implant. In various embodiments, the pathways traced by the circumferential struts 541 may also be variable both in length and localized position relative to the bone axis. In various embodiments, axial struts 542 may be positioned between each adjacent pair of circumferential struts 541 to create uniform windows in each layer and approximately uniform windows throughout the outer mesh 540.

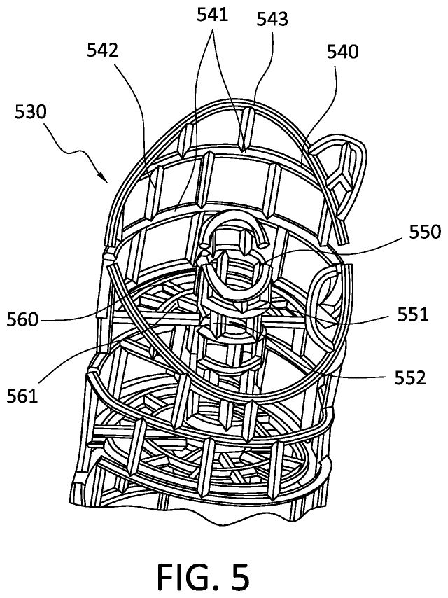

In various embodiments as shown in FIGS. 5 and 6, the inner mesh 550 may be configured to be manually aligned and sized to either match an intramedullary canal or an intramedullary nail. The inner mesh 550 may include circumferential struts 551 that may be used to trace an inner cross-sectional shape of the bone geometry. In various embodiments, the circumferential struts 551 are configured to follow a circular trajectory centered around the bone axis. In various embodiments, the resulting circular structures may share the same inner diameter. The circumferential struts 551 may be equally spaced, using a static spacing parameter, along the axis of the bone, resulting in a series of rings along the length of the implant. In various embodiments, the pathways traced by the circumferential struts 551 may also be variable both in length and localized position relative to the bone axis. In various embodiments, axial struts 552 may be positioned between each adjacent pair of circumferential struts 541 to create uniform windows in each layer and approximately uniform windows throughout the inner mesh 550.

FIG. 6 illustrates in more detail the inner mesh 550. As shown in FIG. 6, the inner mesh 550 may include splines 210 with virtual handles 220 that allow for direct editing and manipulation of the dimensions of the inner mesh 550 to a desired contour of the missing bone portion.

In various embodiments, the interstitial mesh 560, shown in FIG. 5, includes shelves 561 configured to fill the area between the inner mesh 550 and outer mesh 540. The shelves 561 may be equally spaced, using a static parameter, along the axis of the bone. In various embodiments, each shelf 561 includes four quadrants. In various embodiments, the processor may be configured to calculate the distance between the outer mesh 540 and inner mesh 550 and add circumferential struts 541, 551 using a maximum spacing limit, to create uniform rings in each quadrant of each shelf 561. The processor may further be configured to add axial struts 542, 552 between each adjacent pair of circumferential struts 541, 551 in a quadrant, using a maximum spacing limit, to create uniform windows in each quadrant of each shelf 561 and substantially uniform windows in each quadrant of every shelf 561.

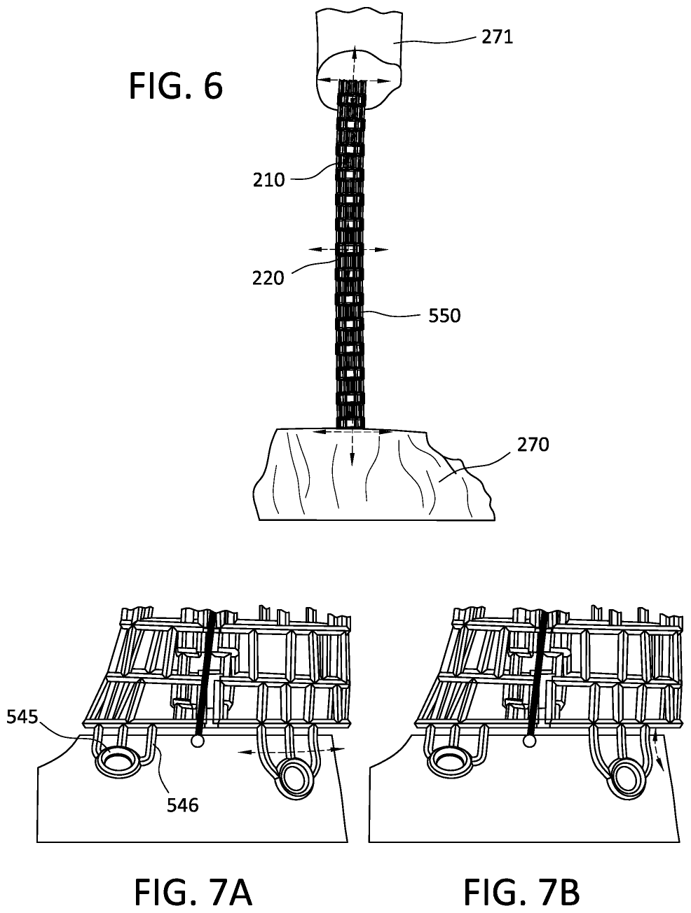

In various embodiments, the processor may further be configured to virtually trim the proximal end 543 of the outer mesh 540 (shown in FIG. 5) or the distal end 544 of the outer mesh 540 (shown in FIGS. 7A and 7B), to optimize the interface between the mesh structure 530 and distal bone fragment 270 and proximal bone fragment 271. In various embodiments, the processor may be configured to generate fixation tabs 545 positioned at the proximal end 543 or the distal end 544 of the outer mesh 540, as shown in FIGS. 7A and 7B. The fixation tabs 545 may extend onto the distal bone fragment 270 or proximal bone fragment 271.

In various embodiments, the processor may further be configured to allow for manipulation of the fixation tabs 545 for optimal positioning on the distal bone fragment 270 or proximal bone fragment 271. In various embodiments, the fixation tabs 545 may be connected to the outer mesh 540 by struts 546. The number of tabs and their locations may be varied as well. This may be done using input from the user or automatically or a combination of both. In various embodiments, the processor may further be configured to include a mapping function that allows for optimal positioning of the struts 546 on the bone surface of the distal bone fragment 270 or proximal bone fragment 271.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A digital long bone reconstruction method, comprising
receiving medical image data of a long bone with a missing section;
displaying on a user interface the image of the long bone;
receiving user input of a first line traversing the long bone indicating a first end of the missing section and a second line traversing the long bone indicating a second end of the missing section;
automatically generating from the image of the long bone, using a processor, a first virtual 3D surface contour of the missing section based upon the received user input having a first geometry and comprising a plurality of editable control regions; and
adjusting at least one of the editable control regions on the first virtual 3D surface contour based on user input to produce a second virtual 3D surface contour of the reconstructed image of the long bone having a second geometry.

2. (canceled)

3. The method of claim 1, wherein the editable control regions comprise a plurality of splines.

4. The method of claim 3, wherein the splines comprise a plurality of manipulation handles.

5. The method of claim 4, wherein the spacing of the plurality of manipulation handles is dependent upon the radius of curvature of the first virtual 3D surface contour.

6. The method of claim 4, wherein the manipulation handles are linked axially along the length of the first virtual 3D surface contour.

7. The method of claim 1, wherein adjusting at least one of the editable control regions comprises dragging the at least one editable control region to an edge of an unreconstructed portion of the bone image.

8. The method of claim 1, wherein the method further comprises printing an implant based upon the second virtual 3D surface contour having the second geometry of the reconstructed image of the long bone to produce an implant; and administering the implant to a patient.

9. The method of claim 8, wherein the second virtual 3D surface contour is printed using a 3D printing device.

10. A digital long bone reconstruction system, comprising:
a user interface configured to receive and display medical image data of a long bone with a missing section from an image capture device; and
a processor coupled to the user interface, the processor configured to:
receive user input of a first line traversing the long bone indicating a first end of the missing section and a second line traversing the long bone indicating a second end of the missing section;
automatically generate from the medical image data of the long bone a first virtual 3D surface contour having a first geometry of the missing section based upon the received user input and comprising a plurality of editable control regions; and
adjust the position of at least one of the editable control regions based on user defined input to produce a second virtual 3D surface contour of the reconstructed image of the long bone having a second geometry.

11. (canceled)

12. The system of claim 10, wherein the editable control regions comprise a plurality of splines.

13. The system of claim 12, wherein the splines comprise manipulation handles.

14. The system of claim 13, wherein the spacing of the plurality of manipulation handles is dependent upon the radius of curvature of the first virtual 3D surface contour.

15. The system of claim 13, wherein the manipulation handles are linked axially along the length of the first virtual 3D surface contour and second virtual 3D surface contour.

16. The system of claim 10, wherein the user input comprises dragging at least one editable control region to an edge of an unreconstructed portion of the bone image.

17. The system of claim 10, wherein the image capture device is configured to capture a three-dimensional (3D) image of the long bone.

18. A digital long bone reconstruction method, comprising
receiving medical image data of a long bone comprising a missing long bone portion;
displaying on a user interface a bone image comprising the missing long bone portion;
receiving user input of a first line traversing the long bone indicating a first end of the missing section and a second line traversing the long bone indicating a second end of the missing section;
automatically generating, using a processor, a first virtual 3D mesh structure contoured to a first geometry of the missing long bone portion based upon the received user input comprising a plurality of editable control regions; and
adjusting at least one of the editable control regions on the first virtual 3D mesh structure based on user input to produce a second virtual 3D mesh structure of the missing long bone portion having a second geometry.

19. The method of claim 18, wherein the editable control regions comprise a plurality of splines.

20. The method of claim 19, wherein the splines comprise manipulation handles.

21. The method of claim 20, wherein the spacing of the plurality of manipulation handles is dependent upon the radius of curvature of the first virtual 3D mesh structure.

22. The method of claim 20, wherein the manipulation handles are linked axially along the length of the first and second virtual 3D mesh structures.

23. The method of claim 18, wherein adjusting at least one of the editable control regions comprises dragging the at least one editable control region to an edge of the missing long bone portion.

24-28. (canceled)

29. The method of claim 18, wherein the method further comprises printing the second virtual 3D mesh structure having the second geometry of the missing long bone portion to produce a mesh implant; and administering the mesh implant to a patient.

30. The method of claim 29, wherein the second virtual 3D mesh structure is printed using a 3D printing device.
